(19) United States
(12) Patent Application Publication
(10) Pub. No.: US 2025/0255237 A1
(43) Pub. Date: Aug. 14, 2025

(54) WATERMELON VARIETY 'E26C.00181'

(71) Applicant: Enza Zaden Beheer B.V., Enkhuizen (NL)

(72) Inventors: Laura Barham Brown, Gilroy, CA (US); Robert Warren Barham, Gilroy, CA (US)

(21) Appl. No.: 19/045,100

(22) Filed: Feb. 4, 2025

Related U.S. Application Data
(60) Provisional application No. 63/551,675, filed on Feb. 9, 2024.

(51) Int. Cl. A01H 6/34; A01H 5/08

(52) U.S. Cl. CPC A01H 6/342; A01H 5/08

(57) ABSTRACT

A new watermelon variety designated ‘E26C.00181’ (‘Big Jack’) is described.

### CROSS-REFERENCE TO RELATED APPLICATIONS

This application claims the benefit of priority to U.S. Provisional Application No. 63/551,675, filed on Feb. 9, 2024, the entire content of which is hereby incorporated by reference.

### FIELD

The present disclosure relates to the field of plants and plant breeding. In particular, this disclosure includes a new watermelon, Citrullus lanatus, variety designated ‘E26C.00181’.

### BACKGROUND

All cultivated forms of watermelon belong to the polymorphic species Citrullus lanatus. As a crop, watermelons are grown commercially wherever environmental conditions permit the production of an economically viable yield. Cultivated watermelons grow as annual plants with large, broad leaves. The leaves may be lobed or simple and are typically orbicular to triangular-ovate in shape. The flowers are monoecious, producing both male and female flowers. The flesh color of cultivated watermelons is red, yellow, or white with ovate to oblong strongly compressed seeds that may be brown or white in color. The characterization of the mature fruit can vary widely among varieties of watermelon. Fruits may be round to oblong or elliptical with rind colors varying from dark green to yellow and rind patterning varying widely.

Many changes that occurred with the domestication of the watermelon relate to fruit morphology, with a specialization in fruit shape, size, and flavor. Cultivated watermelons can vary from 5 to 45 pounds, depending on the variety. In the United States, watermelon is grown in at least 44 states, but the principal watermelon growing states are Georgia, Florida, Texas, and California. Asia is the largest producer of watermelon, with 83.4% of the world production in 2011. Fresh watermelons are consumed in many forms, generally fresh, sliced, or as an ingredient in prepared foods. Over 80% of watermelons grown in the United States are triploid seedless varieties, preferred for their ease of consumption, increased yield, and premium market value over diploid seeded varieties.

Citrullus lanatus is a member of the family Cucurbitaceae, which contains about 90 genera and 700 to 760 species. The family includes melons, pumpkins, squashes, gourds, cucumber, loofah, and many weeds. The genus Citrullus, to which the watermelon belongs, consists of about four species, including C. colocynthis, C. rehmii, and C. ecirrhosus, all of which may be crossed with each other successfully. The watermelon is believed to be native to southern Africa and has been cultivated there for about 4000 years.

Regular, seeded watermelon is diploid and has eleven pairs of chromosomes (2n=2x=22). There also exist tetraploid varieties, which have 44 chromosomes in their somatic cells (2n=4x=44). Popular “seedless” varieties are triploid, meaning they have 33 chromosomes in their somatic cells (3x=33) and are derived from a cross between male diploid and female tetraploid parents. When triploid plants are grown in the presence of diploid plants, the triploid plants produce seedless fruit. These seedless varieties may sometimes produce fruit with small, edible white ovules (pips), similar to those in immature cucumbers.

Watermelon is an important and valuable field crop with an array of observable and detectable traits of importance both to watermelon consumers and watermelon growers. Traits of importance include taste, texture, size, rind patterns, and shapes. Watermelon consumers desire watermelons that have excellent taste and sweetness. In addition, the texture and firmness of flesh is critical to the consumer experience of eating watermelons. Watermelon fruit comes in a wide range of sizes, from as small as one kilogram up to twenty or more kilograms, with the main commercial range being from approximately three kilograms up to approximately twelve kilograms. There are dozens of rind patterns possible on watermelon fruit. While consumers tend to prefer particular rind patterns, novel rind patterns can also lead to fruit purchase. Watermelon fruit can have an array of shapes from round to oval (length/width ratios up to 3:1). Consumers have distinct preferences for fruit shape. Growers also can have preferences for fruit shape as, for example, in some markets a blocky shape is considered easier to ship than a round shape. Growers also are highly focused on having high yields from their watermelon fields. At the same time, growers must meet quality standards for their crops to be accepted by wholesale buyers and shippers, including producing fruit free of defects such as hollow-heart, internal growth, not having “seeds” in the seedless triploid hybrids, sufficiently good color, and good appearance of fruit. Moreover, for both quality and yield, growers often need certain disease resistances for their watermelon varieties.

In spite of many genetic improvements to watermelons for both growing and consumption, the consumer experience of watermelons is often disappointing. This is largely due to watermelon being a commodity in the marketplace, with watermelons coming from many different growers and being sold based on a wholesale price rather than taste quality. In addition, current watermelon breeding is focused on producing watermelons that look similar to other watermelons in the marketplace.

### SUMMARY

There is a need to provide watermelon varieties with improved taste that have distinguishing features (such as higher yields, larger fruit sizes, distinct rind patterns or distinct fruit shapes), which will satisfy grower requirements and provide easily recognizable and better-tasting watermelons for consumers.

In order to meet these needs, the present disclosure provides, among other things, watermelon characterized by improved taste and distinguishing features, such as high yield, blocky-oval fruit shape, dark crimson rind pattern, and flesh that is firm, sweet, and red. In addition, the present disclosure is directed to, among other things, watermelon varieties with improved internal quality, and/or more desirable fruit sizes and pest resistances.

The present disclosure provides a triploid hybrid watermelon (Citrullus lanatus (Thunb.) Matsum. et Nakai) variety seed designated as ‘E26C.00181’ (i.e., ‘Big Jack’) having NCIMB Accession Number X1. In some embodiment, the present disclosure is directed to a Citrullus lanatus watermelon plant and/or parts isolated therefrom produced by growing ‘E26C.00181’ watermelon seed (which plants and parts can be referred to, e.g., as ‘E26C.00181’ plants and ‘E26C.00181’ parts, respectively). In some embodiment, the present disclosure is directed to a Citrullus lanatus seed, plants grown from the seed having all, or essentially all, of the physiological and morphological characteristics of a Citrullus lanatus plant produced by growing ‘E26C.00181’ watermelon seed having NCIMB Accession Number X1, and/or parts isolated therefrom.

Watermelon plant parts include watermelon leaves, ovules, pollen, seeds, watermelon fruits, parts of watermelon fruits, flowers, scions, rootstocks, roots, root tips, stems, hypocotyls, petioles, cotyledons, cuttings, anthers, pistils, stalks, meristems, protoplasts, cells, and/or the like. In some embodiment, the present disclosure is directed to watermelon leaves, ovules, pollen, seeds, watermelon fruits, parts of watermelon fruits, flowers, scions, rootstocks, roots, root tips, stems, hypocotyls, petioles, cotyledons, cuttings, anthers, pistils, stalks, meristems, protoplasts, and/or cells isolated from ‘E26C.00181’ watermelon plants. In certain embodiments, the present disclosure is directed to pollen grains or ovules isolated from ‘E26C.00181’ watermelon plants. In some embodiment, the present disclosure is directed to protoplasts produced from ‘E26C.00181’ watermelon plants. In some embodiment, the present disclosure is directed to tissue and/or culture produced from protoplasts or regenerable cells of ‘E26C.00181’ watermelon plants, wherein said regenerable cells or protoplasts are produced from a plant part selected from the group consisting of root, root tip, meristematic cell, stem, hypocotyl, petiole, cotyledon, leaf, flower, anther, pollen, pistil, stalk, and fruit. The present disclosure is directed to watermelon plants regenerated from the tissue culture, where the plants regenerated from the tissue culture have all, or essentially all, of the morphological and physiological characteristics of ‘E26C.00181’ watermelon.

In some embodiment, the present disclosure is directed to a method of producing seedless watermelon fruit, the method including: (a) crossing a first ‘E26C.00181’ watermelon plant, representative sample of seed having been deposited under NCIMB Accession Number X1, with a second diploid watermelon plant; (b) allowing seedless fruit to form; and (c) harvesting the seedless fruit.

In some embodiment, the present disclosure provides methods of vegetatively propagating a plant of watermelon variety ‘E26C.00181’, the method including the steps of: (a) collecting tissue capable of being propagated from a plant of watermelon variety ‘E26C.00181’, representative seed of said watermelon variety ‘E26C.00181’ having been deposited under NCIMB Accession Number X1; and (b) producing a rooted plant from said tissue.

According to the disclosure, there is provided a watermelon plant designated ‘E26C.00181’. This disclosure thus relates to the plants of watermelon ‘E26C.00181’, the plant parts of watermelon ‘E26C.00181’, and to the seeds of watermelon ‘E26C.00181’. This disclosure also relates to methods for producing other watermelon cultivars or hybrids derived from watermelon ‘E26C.00181’ and to the watermelon cultivars and hybrids derived by the use of those methods.

In some embodiments, the present disclosure provides methods of introducing a trait into watermelon variety ‘E26C.00181’, the method including: (a) utilizing as a recurrent parent a plant of watermelon variety ‘E26C.00181’ (e.g., a plant produced by growing a watermelon seed designated as ‘E26C.00181’) by crossing the recurrent parent plant with a donor plant that comprises the trait, thereby producing F1 progeny; (b) selecting an F1 progeny that comprises the trait; (c) backcrossing the selected F1 progeny with a plant of the same line or variety used as the recurrent parent in step (a) to produce backcross progeny; (d) selecting a backcross progeny comprising the trait and otherwise comprising all or essentially all of the morphological and physiological characteristics of the recurrent parent plant; and (e) repeating steps (c) and (d) for at least one additional generation (e.g., for an additional 3-10 generations), thereby producing further backcross progeny. In some embodiment, backcross progeny have the introduced trait and otherwise have all, or essentially all, of the morphological and physiological characteristics of watermelon variety ‘E26C.00181’, or a seed thereof.

In some embodiment, the present disclosure provides methods of producing a plant of, or derived from, watermelon line ‘E26C.00181’ that includes an added or further trait. In some embodiments, the present disclosure provides a method of producing a watermelon plant with an added trait, where the method includes introducing a transgene conferring the trait into a plant of watermelon line ‘E26C.00181’, and a sample of seed of said line ‘E26C.00181’ has been deposited under NCIMB Accession Number X1. In some embodiments, the produced watermelon plant has the trait and otherwise has all, or essentially all, of the morphological and physiological characteristics of watermelon variety ‘E26C.00181’, or a seed thereof.

In some embodiment, the present disclosure provides a watermelon plant having all, or essentially all, of the morphological and physiological characteristics of watermelon variety ‘E26C.00181’ and further including a transgene.

In some embodiment, the present disclosure is directed to single gene converted plants of watermelon ‘E26C.00181’. The single transferred gene may preferably be a dominant or recessive allele. Preferably, the single transferred gene will confer such trait as sex determination, herbicide resistance, insect resistance, resistance for bacterial, fungal, or viral disease, improved harvest characteristics, enhanced nutritional quality, and/or improved agronomic quality. The single gene may be a naturally occurring watermelon gene or a transgene introduced through genetic engineering techniques.

In some embodiment, the present disclosure is directed to methods for developing watermelon plants in a watermelon plant breeding program using plant breeding techniques including recurrent selection, backcrossing, pedigree breeding, restriction fragment length polymorphism enhanced selection, genetic marker enhanced selection, and/or transformation. Marker loci such as restriction fragment polymorphisms or random amplified DNA have been published for many years and may be used for selection (See, Pierce et al., HortScience (1990) 25:605-615; Wehner, T., Cucurbit Genetics Cooperative Report, (1997) 20:66-88; and Kennard et al., Theoretical Applied Genetics (1994) 89:217-224). Seeds, watermelon plants, and parts thereof produced by such breeding methods are also part of the disclosure.

In some embodiments, the present disclosure is directed to watermelon seeds resulting from methods of making a watermelon variety of the present disclosure. In additional embodiments, the present disclosure is directed to watermelon plants, and/or parts thereof, obtained from growing the seeds of the present disclosure. In additional embodiments, the present disclosure is directed to watermelon plants, and/or parts thereof, having all, or essentially all, of the physiological and morphological characteristics of the watermelon plants of the present disclosure. In additional embodiments, the present disclosure is directed to watermelon tissue culture, obtained from the plants of the present disclosure. In some embodiments, the tissue culture of the present disclosure is produced from a plant part selected from the group consisting of leaf, anther, pistil, stem, petiole, root, root tip, fruit, seed, flower, cotyledon, hypocotyl, embryo, and meristematic cell.

In addition to the exemplary aspects and embodiments described above, further aspects and embodiments will become apparent by reference by study of the following descriptions.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1 shows whole fruit of watermelon variety ‘E26C.00181’ and longitudinal cross-sections of fruit of watermelon variety ‘E26C.00181’.

### DETAILED DESCRIPTION

The present disclosure is directed towards watermelon varieties exhibiting desired rind, flesh, size, and shape characteristics. These desired rind, flesh, size, and shape characteristics can be accomplished by crossing two watermelon plants such that the progeny watermelon plants exhibit rind, flesh, size, and shape characteristics of both parent plants. In an exemplary embodiment, the present disclosure is directed towards triploid, seedless, watermelon plants.

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possesses the traits to meet the program goals. The selected germplasm is crossed in order to recombine the desired traits, and then through selection, varieties or parent lines are developed. The goal is to combine in a single variety or hybrid an improved combination of desirable traits from the parental germplasm. These important traits may include, among other things, higher yield, field performance, resistance to diseases and insects, and/or tolerance to drought and heat. These important traits may also include fruit and agronomic quality such as fruit shape, fruit appearance, fruit flesh firmness, and/or internal quality of fruit.

Choice of breeding or selection methods can depend on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and/or recurrent selection.

The complexity of inheritance influences choice of the breeding method. Backcross breeding is used to transfer one or a few favorable genes for a highly heritable trait into a desirable cultivar. This approach has been used extensively for breeding disease-resistant varieties. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program may include a periodic, objective evaluation of the efficiency of the breeding procedure. Evaluation criteria vary depending on the goal and objectives, and can include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to appropriate standards in environments representative of the commercial target area(s) for at least three years. The best lines can then be candidates for new commercial cultivars. Those still deficient in a few traits may be used as parents to produce new populations for further selection. These processes, which lead to the final step of marketing and distribution, may take from ten to twenty years from the time the first cross or selection is made.

One goal of watermelon plant breeding is to develop new, unique, and genetically superior watermelon cultivars and hybrids. A breeder can initially select and cross two or more parental lines, followed by repeated selfing and selection, producing many new genetic combinations. A plant breeder can then select which germplasms to advance to the next generation. These germplasms may then be grown under different geographical, climatic, and soil conditions, and further selections can be made during, and at the end of, the growing season.

The development of commercial watermelon cultivars thus requires the development and/or selection of watermelon parental lines, the crossing of these lines, and the evaluation of the crosses. Pedigree breeding and recurrent selection breeding methods may be used to develop cultivars from breeding populations. Breeding programs can be used to combine desirable traits from two or more varieties or various broad-based sources into breeding pools from which lines are developed by selfing and selection of desired phenotypes. The new lines are crossed with other lines and the hybrids from these crosses are evaluated to determine which have commercial potential.

Pedigree breeding is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

New varieties can also be developed from more than two parents. The technique, known as modified backcrossing, uses different recurrent parents during the backcrossing. Modified backcrossing may be used to replace the original recurrent parent with a variety having certain more desirable characteristics or multiple parents may be used to obtain different desirable characteristics from each.

In addition to being used to create a backcross conversion, backcrossing can also be used in combination with pedigree breeding. As discussed previously, backcrossing can be used to transfer one or more specifically desirable traits from one variety, the donor parent, to a developed variety called the recurrent parent, which has overall good agronomic characteristics yet lacks that desirable trait or traits. However, the same procedure can be used to move the progeny toward the genotype of the recurrent parent, but at the same time retain many components of the nonrecurrent parent by stopping the backcrossing at an early stage and proceeding with selfing and selection. For example, an inbred watermelon line may be crossed with another variety to produce a first generation progeny plant. The first generation progeny plant may then be backcrossed to one of its parent varieties to create a BC1 or BC2. Progeny are selfed and selected so that the newly developed variety has many of the attributes of the recurrent parent and yet several of the desired attributes of the nonrecurrent parent. This approach leverages the value and strengths of the recurrent parent for use in new watermelon varieties.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques known in the art that are available for the analysis, comparison and characterization of plant genotype. Such techniques include, without limitation, High Resolution Melting (HRM), DNA- or RNA-sequencing, CAPS Markers, ELISA, Western blot, microarrays, Single Nucleotide Polymorphisms (SNPs), Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Differential Display Polymerase Chain Reaction (DD-PCR), Quantitative Real-Time Polymerase Chain Reaction (qRT-PCR), DNA Amplification Fingerprinting (DAF), Sequence Characterized Amplified Regions (SCARs), Amplified Fragment Length Polymorphisms (AFLPs), and Simple Sequence Repeats (SSRs, which are also referred to as Microsatellites).

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding may also be used to introduce new traits into watermelon varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid, or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

Additional non-limiting examples of breeding methods that may be used include, without limitation, those found in Principles of Plant Breeding, John Wiley and Son, pp. 115-161, 1960; Allard, 1960; Simmonds, 1979; Sneep et al., 1979; Fehr, 1987; “Carrots and Related Vegetable Umbelliferae”, Rubatzky, V. E., et al., 1999.

### Definitions

In the description that follows, a number of terms are used. In order to provide a clear and consistent understanding of the specification and claims, including the scope to be given such terms, the following definitions are provided:

Allele. The allele is any of one or more alternative forms of a gene, all of which relate to one trait or characteristic. In a diploid cell or organism, the two alleles of a given gene occupy corresponding loci on a pair of homologous chromosomes.

Androecious plant. A plant having staminate flowers only.

Anthracnose. Anthracnose of cucurbits is a disease caused by the fungus Colletotrichum orbiculare (also called Colletotrichum lagenarium). Infected plants have lesions on leaves, stems, and fruits.

Backcrossing. Backcrossing is a process in which a breeder repeatedly crosses hybrid progeny back to one of the parents, for example, a first generation hybrid F₁ with one of the parental genotype of the F₁ hybrid.

Blossom end. The blossom end is the distal end of the fruit (the “far” end as measured from the base of the plant) where the flower blossom is located. The other end of a fruit is the stem end.

Blossom scar. The blossom scar is the small mark left on the distal end of the fruit after the flower falls off.

Covered cultivation. Any type of cultivation where the plants are not exposed to direct sunlight. The covering includes but is not limited to greenhouses, glasshouses, nethouses, plastic houses, and tunnels.

Essentially all the physiological and morphological characteristics. A plant having essentially all the physiological and morphological characteristics of another plant means that the plants share essentially all physiological and morphological characteristics identified herein, except, e.g., where applicable, with respect to characteristics derived from a converted gene that differs between the plants as a result backcrossing, mutation, or genetic engineering.

Fusarium wilt. Fusarium wilt of watermelon is a disease caused by the fungus Fusarium oxysporum f. sp. niveum (FON). Infected plants wilt, have brown-streaked vascular tissue, and produce small and misshapen fruit.

Gene. As used herein, “gene” refers to a segment of nucleic acid. A gene can be introduced into a genome of a species, whether from a different species or from the same species, using transformation or various breeding methods.

Gynoecious plant. A plant having pistillate flowers only.

Monoecious plant. A plant having separate staminate and pistillate flowers on the same plant.

Percent Identity. Percent identity as used herein refers to the comparison of the homozygous alleles of two watermelon lines, hybrids or varieties. Percent identity is determined by comparing a statistically significant number of the homozygous alleles of two developed varieties, lines or hybrids. For example, a percent identity of 90% between watermelon plant 1 and watermelon plant 2 means that the two plants have the same allele at 90% of their loci.

Propagate. To “propagate” a plant means to reproduce the plant by means including, but not limited to, seeds, cuttings, divisions, tissue culture, embryo culture, or other in vitro method.

Quantitative Trait Loci (QTL). Quantitative trait loci refer to genetic loci that control to some degree numerically representable traits that are usually continuously distributed.

Regeneration. Regeneration refers to the development of a plant from tissue culture.

Seedless. Seedless plants refer to triploid watermelon plants that produce fruit without viable seeds. These fruit may be devoid of seeds or seed-like structures, or may contain immature white ovules.

Single gene converted. Single gene converted or conversion plant refers to plants which are developed by a plant breeding technique called backcrossing wherein essentially all of the desired morphological and physiological characteristics of an inbred are recovered in addition to the single gene transferred into the inbred via the backcrossing technique, genetic engineering, or mutation.

Transgene. A “transgene” is a gene taken or copied from one organism and inserted into another organism. A transgene may be a gene that is foreign to the receiving organism or it may be a modified version of a native, or endogenous, gene.

### Overview of the Variety ‘E26C.00181’

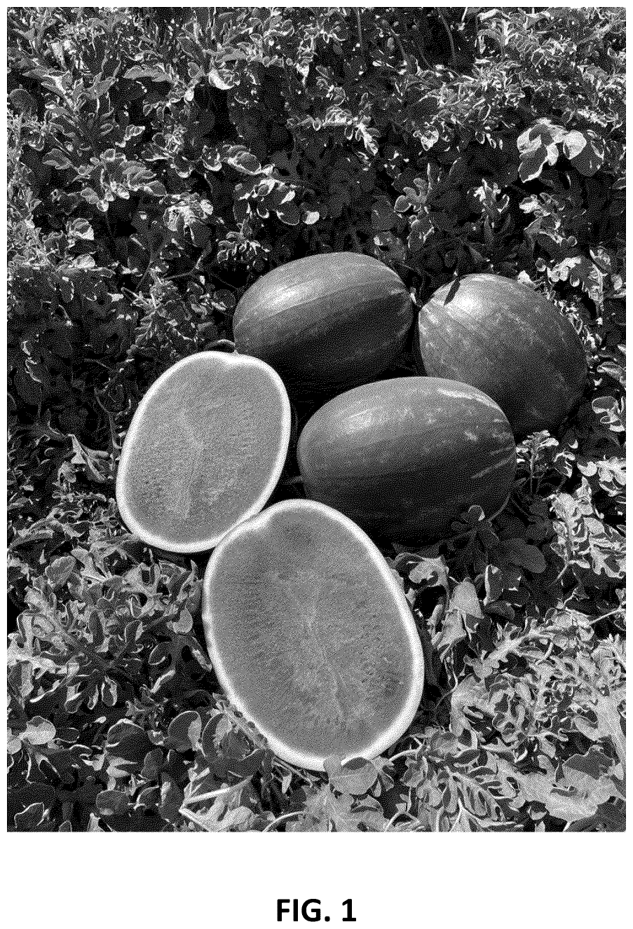

Watermelon variety ‘E26C.00181’ is a triploid watermelon variety that produces blocky-oval shaped fruit with dark green stripes on medium green colored skin. ‘E26C.00181’ is suitable for cultivation in the field. ‘E26C.00181’ is useful for fresh market and processed products. Additionally, watermelon variety ‘E26C.00181’ is moderately resistant to Anthracnose (Colletotrichum orbiculare) Race 1, Fusarium wilt (Fusarium oxysporum f. sp. niveum) Race 1, and sunburn. FIG. 1 depicts fruits of watermelon variety ‘E26C.00181’. Watermelon variety ‘E26C.00181’ is the result of numerous generations of plant selections chosen for, and characterized by, high yield, flesh that is firm, sweet, and red, and intermediate resistance to Anthracnose Race 1 and Fusarium Race 1, and/or by a subset thereof (e.g., by a combination of two or more such characteristics that is desirable for production and/or marketability). ‘E26C.00181’ is also chosen for its dark crimson rind pattern, large fruit size, small pips, and/or by a combination thereof (e.g., by a combination thereof that is desirable for production and/or marketability), as compared to various other dark crimson hybrid watermelon varieties available in the market. The large fruit size is advantageous for growth of ‘E26C.00181’ under conditions that cause stress in watermelon plants.

The variety has shown uniformity and stability for the traits, within the limits of environmental influence for the traits. No variant traits have been observed or are expected in ‘E26C.00181’.

### Objective Description of the Variety ‘E26C.00181’

In various embodiments, watermelon variety ‘E26C.00181’ can be characterized by the following morphologic and other characteristics. The triploid watermelons of ‘E26C.00181’ were generally planted as a transplant and not as a seed. Exemplary crop measurements shown below for ‘E26C.00181’ were made 84 days after planting in California, USA.

General fruit type: Blocky large

Use: Commercial fruit production variety

Maturity:
Number of days from emergence to anthesis: 39 days
Number of days from pollination to maturity: 45 days
Number of days to relative maturity from sowing: 84 days
Maturity category: Medium

Ploidy: Triploid

Plant:

Cotyledon shape: Flat

Plant sex form: Monoecious

Number of flowers at first fruit set:
Staminate: dozens
Pistillate: 5

Stem:
Shape in cross-section: Round
Surface: Pubescent

Leaf:
Shape: Ovate
Lobes: Lobed
Dorsal surface pubescence: Present
Dorsal surface color: Medium green
Ventral surface pubescence: Present
Ventral surface color: Medium green
Blistering: Weak
Vine color: Green

Flower:
Staminate flower diameter: 2.5 cm
Pistillate flower diameter: 2.5 cm
Color: Yellow

Mature Fruit:
Shape: Oval
Length: 31 cm
Diameter at midsection: 24 cm
Average weight: 8.5 kg
Maximum weight: 10.5 kg
Fruit surface: Smooth
Waxy layer: Present
Skin color pattern: Mottle/net
Primary color (ground color): Medium green
Secondary color: Dark green
Conspicuousness of veining: Weak
Pattern of stripes: Only one colored
Width of stripes: Very broad
Main color of stripes: Dark green
Conspicuousness of stripes: Strong
Sharpness of margin of stripes: Diffuse

Rind:
Texture: Tough
Thickness of blossom end: 15 mm
Thickness of sides: 15 mm

Flesh:
Texture: Crisp
Coarseness: Fine (little fiber)
Color: RHS 44B (Dark red)

Chemistry, composition, and characteristics of ripe fruit:
Soluble solids of juice (center of fruit by refractometer): 12.5%
Hollow heart: 0%
Placental separation: 0%
Transverse crack: 0%
Description of ripe fruit: Crunchy flesh

Seeds used to grow plants of the candidate variety:
Size: Medium
Length: 10 mm
Seed width: 6 mm
Thickness: 3 mm
Weight per 1000 seeds: 75 grams
Number of seeds per fruit: 75
Color: Dark brown mottled

Growth conditions:
Type of culture: Field
Main use(s): Fresh market and Processed products
Mechanical harvest: Not suitable
Suitable growing regions: Global

Disease and stress resistance:
Anthracnose (Colletotrichum orbiculare) (Co) Race 1: Moderately resistant Fusarium wilt (Fusarium oxysporum f. sp. niveum) (Fon) Race 1: Moderately resistant
Sunburn: Moderately resistant

### Comparisons to Other Watermelon Varieties

Table 1 below compares some of the characteristics of the triploid watermelon variety ‘E26C.00181’ with the commercial triploid watermelon variety ‘Cracker Jack’ (Enza Zaden) (protected by U.S. patent application Ser. No. 16/934,752 and U.S. Pat. No. 11,470,797) and watermelon variety ‘Fascination’ (Syngenta). Column 1 lists the characteristics, column 2 shows the characteristics for triploid watermelon variety ‘E26C.00181’, column 3 shows the characteristics for commercial triploid watermelon variety ‘Cracker Jack’, and column 4 shows the characteristics for commercial triploid watermelon variety, ‘Fascination’.

TABLE 1

| Characteristic | ‘E26C.00181’ | ‘Cracker Jack’ | ‘Fascination’ |
|---|---|---|---|
| Pip size | Small | Medium | |
| (undeveloped ovule) | | | |
| Fruit main color of | Very dark green | | Medium green |
| stripes | | | |

### Further Embodiments

This disclosure also is directed to methods for producing a watermelon plant by crossing a first diploid parent watermelon plant with a second tetraploid parent watermelon plant to produce a triploid offspring watermelon plant. In some embodiments, the triploid offspring plant has a dark mottled stripe rind pattern, bright red flesh color, strong vine, medium-large fruit size, oval-blocky fruit shape, small pips within the fruit, and concentrated fruit set. In some embodiments, the triploid offspring plant is ‘E26C.00181’.

Occasionally, triploid watermelon varieties produce true and viable seed at a very low frequency. This is due to the random segregation of chromosomes, which can result in chromosomes occasionally ending up distributed back to normal in the ovule and pollen such that they are balanced enough to be viable. Thus, occasional viable pollen from a triploid variety can be “bridged”, or crossed, onto a normal diploid or tetraploid line to make viable hybrid seed. Similarly, occasional viable ovules within a triploid can be bridged or crossed onto, using pollen from a normal tetraploid or diploid line to create viable seed. Also occasionally, a triploid within and of itself can make a viable seed that can make a fertile plant. This occasional fertility of triploid varieties is well-known by watermelon breeders, and can be taken advantage of to incorporate triploid varieties with desirable characteristics into breeding programs. Watermelon variety ‘E26C.00181’, like other triploid varieties, occasionally produces flowers with viable pollen or viable ovules. Similarly, watermelon variety ‘E26C.00181’ occasionally produces fruit containing viable, fertile seed. An additional aspect of this disclosure is directed to methods for producing offspring of watermelon variety ‘E26C.00181’ (e.g., using viable pollen, viable ovules, or viable seed occasionally produced by ‘E26C.00181’).

### Genetic Marker Profile Through SSR and First Generation Progeny

In addition to phenotypic observations, a plant can also be identified by its genotype. The genotype of a plant can be characterized through a genetic marker profile which can identify plants of the same variety or a related variety or be used to determine or validate a pedigree. There are many further laboratory-based techniques known in the art that are available for the analysis, comparison and characterization of plant genotype. Such techniques include, without limitation, High Resolution Melting (HRM), DNA- or RNA-sequencing, CAPS Markers, ELISA, Western blot, microarrays, Single Nucleotide Polymorphisms (SNPs), Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Differential Display Polymerase Chain Reaction (DD-PCR), Quantitative Real-Time Polymerase Chain Reaction (qRT-PCR), DNA Amplification Fingerprinting (DAF), Sequence Characterized Amplified Regions (SCARs), Amplified Fragment Length Polymorphisms (AFLPs), and Simple Sequence Repeats (SSRs, which are also referred to as Microsatellites).

Particular markers used for these purposes are not limited to any particular set of markers, but are envisioned to include any type of marker and marker profile which provides a means of distinguishing varieties.

The present disclosure includes a watermelon plant characterized by molecular and physiological data obtained from the representative sample of said variety deposited with the National Collection of Industrial, Food and Marine Bacteria Ltd. (NCIMB Ltd.). Further provided by the disclosure is a watermelon plant formed by the combination of the disclosed watermelon plant or plant cell with another watermelon plant or cell and including the homozygous alleles of the variety.

Means of performing genetic marker profiles using SSR polymorphisms are well known in the art. SSRs are genetic markers based on polymorphisms in repeated nucleotide sequences, such as microsatellites. A marker system based on SSRs can be highly informative in linkage analysis relative to other marker systems in that multiple alleles may be present. Another advantage of this type of marker is that, through use of flanking primers, detection of SSRs can be achieved, for example, by polymerase chain reaction (PCR), thereby eliminating the need for labor-intensive Southern hybridization. PCR detection is done by use of two oligonucleotide primers flanking the polymorphic segment of repetitive DNA. Repeated cycles of heat denaturation of the DNA followed by annealing of the primers to their complementary sequences at low temperatures, and extension of the annealed primers with DNA polymerase, include the major part of the methodology.

Following amplification, markers can be scored by electrophoresis of the amplification products. Scoring of marker genotype is based on the size of the amplified fragment, which may be measured by the number of base pairs of the fragment. While variation in the primer used or in laboratory procedures can affect the reported fragment size, relative values should remain constant regardless of the specific primer or laboratory used. When comparing varieties it is preferable if all SSR profiles are performed in the same lab.

### Single-Gene Conversions

When the terms watermelon plant, cultivar, hybrid, or watermelon line are used in the context of the present disclosure, this also includes any single gene conversions of that line. The term “single gene converted plant” as used herein refers to those watermelon plants which are developed by a plant breeding technique called backcrossing wherein essentially all of the desired morphological and physiological characteristics of a cultivar are recovered in addition to the single gene transferred into the line via the backcrossing technique. Backcrossing methods can be used with the present disclosure to improve or introduce a characteristic into the line. The term “backcrossing” as used herein refers to the repeated crossing of a hybrid progeny back to one of the parental watermelon plants for that line, backcrossing 1, 2, 3, 4, 5, 6, 7, 8, or more times to the recurrent parent. The parental watermelon plant which contributes the gene for the desired characteristic is termed the nonrecurrent or donor parent. This terminology refers to the fact that the nonrecurrent parent is used one time in the backcross protocol and therefore does not recur. The parental watermelon plant to which the gene or genes from the nonrecurrent parent are transferred is known as the recurrent parent as it is used for several rounds in the backcrossing protocol (Poehlman & Sleper, 1994; Fehr, 1987). In a typical backcross protocol, the original cultivar of interest (recurrent parent) is crossed to a second line (nonrecurrent parent) that carries the single gene of interest to be transferred. The resulting progeny from this cross are then crossed again to the recurrent parent and the process is repeated until a watermelon plant is obtained wherein essentially all of the desired morphological and physiological characteristics of the recurrent parent are recovered in the converted plant, in addition to the single transferred gene from the nonrecurrent parent.

The selection of a suitable recurrent parent is an important step for a successful backcrossing procedure. The goal of a backcross protocol is to alter or substitute a single trait or characteristic in the original line. To accomplish this, a single gene of the recurrent cultivar is modified or substituted with the desired gene from the nonrecurrent parent, while retaining essentially all of the rest of the desired genetic, and therefore the desired physiological and morphological, constitution of the original line. The choice of the particular nonrecurrent parent will depend on the purpose of the backcross; one of the major purposes is to add some commercially desirable, agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many single gene traits have been identified that are not regularly selected for in the development of a new line but that can be improved by backcrossing techniques. Single gene traits may or may not be transgenic, and examples of these traits include but are not limited to, male sterility, modified fatty acid metabolism, modified carbohydrate metabolism, herbicide resistance, resistance for bacterial, fungal, or viral disease, insect resistance, enhanced nutritional quality, industrial usage, yield stability, and yield enhancement. These genes are generally inherited through the nucleus. Several of these single gene traits are described in U.S. Pat. Nos. 5,777,196, 5,948,957 and 5,969,212, the disclosures of which are specifically hereby incorporated by reference.

### Tissue Culture

As used herein, the term “tissue culture” indicates a composition including isolated cells of the same or a different type or a collection of such cells organized into parts of a plant. Exemplary types of tissue cultures are protoplasts, calli, meristematic cells, and regenerable plant cells that can generate tissue culture that are intact in plants or parts of plants, such as leaves, pollen, embryos, roots, root tips, anthers, pistils, flowers, seeds, petioles, stalks, and the like. Means for preparing and maintaining plant tissue culture are well known in the art. By way of example, a tissue culture including organs has been used to produce regenerated plants. The use of tissue culture to propagate watermelon plants is exemplified in Adelberg, J. W., B. B. Rhodes, Micropropagation from zygotic tissue of watermelon, C. E. Thomas (ed.) Proc. of the Cucurbitaceae 89: Evaluation and enhancement of cucurbit germplasm, Charleston S.C., USA; and Zhang et al., Shoot regeneration from immature cotyledon of watermelon, Cucurbit Genetics Coop. 17:111-115 (1994).

Transformation of plant protoplasts also can be achieved using methods based on calcium phosphate precipitation, polyethylene glycol treatment, electroporation, and combinations of these treatments (see, e.g., Potrykus et al., 1985; Omirulleh et al., 1993; Fromm et al., 1986; Uchimiya et al., 1986; Marcotte et al., 1988). Transformation of plants and expression of foreign genetic elements is exemplified in Choi et al. (1994) and Ellul et al. (2003). Direct uptake of DNA into protoplasts using CaCl2 precipitation, polyvinyl alcohol, or poly-L-ornithine has also been reported (see, e.g., Hain et al., 1985 and Draper et al., 1982). Electroporation of protoplasts and whole cells and tissues have also been described (see, e.g., Saker et al., 1998; Donn et al., 1990; D'Halluin et al., 1992; and Laursen et al., 1994; Chupean et al., 1989).

### Additional Breeding Methods

This disclosure also is directed to methods for producing a triploid watermelon plant by crossing a tetraploid parent watermelon plant with a diploid parent watermelon plant to generate a triploid watermelon plant. In this manner, a diverse array of watermelon rind patterns, watermelon flesh color, and watermelon taste may be developed and selected for superior qualities including appearance and taste. Further, watermelon varieties may be developed and selected for superior qualities including improved firmness of flesh, improved internal quality, increased yield, and hardiness. Breeding steps that may be used in the watermelon plant breeding program include pedigree breeding, backcrossing, mutation breeding, and recurrent selection. In conjunction with these steps, techniques such as RFLP-enhanced selection, genetic marker enhanced selection (for example SSR markers) and the making of double haploids may be utilized.

The cultivar of the disclosure can also be used for transformation where exogenous genes are introduced and expressed by the cultivar of the disclosure. Genetic variants of ‘E26C.00181’ created either through traditional breeding methods or through transformation of watermelon variety ‘E26C.00181’ by any of a number of protocols known to those of skill in the art are intended to be within the scope of this disclosure.

Mutations for use in mutation breeding can be induced in plants by using mutagenic chemicals such as ethyl methane sulfonate (EMS), by irradiation of plant material with gamma rays or fast neutrons, or by other means. The resulting nucleotide changes are random, but in a large collection of mutagenized plants the mutations in a gene of interest can be readily identified by using the TILLING (Targeting Induced Local Lesions IN Genomes) method (McCallum et al. (2000) Targeted screening for induced mutations. Nat. Biotechnol. 18, 455-457, and Henikoff et al. (2004) TILLING. Traditional mutagenesis meets functional genomics. Plant Physiol. 135, 630-636). The principle of this method is based on the PCR amplification of the gene of interest from genomic DNA of a large collection of mutagenized plants in the M2 generation. By DNA sequencing or by looking for point mutations using a single-strand specific nuclease, such as the CEL-I nuclease (Till et al. (2004) Mismatch cleavage by single-strand specific nucleases. Nucleic Acids Res. 32, 2632-2641), the individual plants that have a mutation in the gene of interest are identified. By screening many plants, a large collection of mutant alleles is obtained, each giving a different effect on gene expression or enzyme activity. The gene expression or protein levels can for example be tested by transcript analysis levels (e.g., by RT-PCR) or by quantification of protein levels with antibodies. Plants with the desired reduced gene expression or reduced protein expression are then back-crossed or crossed to other breeding lines to transfer only the desired new allele into the background of the crop wanted.

Genes of interest for use in breeding may also be edited using gene editing techniques including transcription activator-like effector nuclease (TALEN) gene editing techniques, clustered Regularly Interspaced Short Palindromic Repeat (CRISPR/Cas9) gene editing techniques, and/or zinc-finger nuclease (ZFN) gene editing techniques. For this, transgenic plants are generated expressing one or more constructs targeting the gene of interest. These constructs may include, without limitation, an anti-sense construct, an optimized small-RNA construct, an inverted repeat construct, a targeting construct, a guide RNA construct, a construct encoding a targeting protein, and/or a combined sense-anti-sense construct, and may work in conjunction with a nuclease, an endonuclease, and/or an enzyme, so as to downregulate the expression of a gene of interest.

One of ordinary skill in the art of plant breeding would know how to evaluate the traits of two plant varieties to determine if there is no significant difference between the two traits expressed by those varieties. For example, see Fehr and Walt, Principles of Cultivar Development, p. 261-286 (1987). Thus the disclosure includes watermelon plants including a combination of at least two traits selected from the combination of traits listed in the Objective Description of the Variety ‘E26C.00181’, so that said progeny watermelon plant is not significantly different for said traits than watermelon ‘E26C.00181’, as determined at the 5% significance level when grown in the same environmental conditions and/or may be characterized by percent similarity or identity to hybrid watermelon ‘E26C.00181’, as determined by SSR markers or another genotyping method. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as firmness of flesh, internal quality, taste, appearance, yield, disease resistance, pest resistance, and plant performance in extreme environmental conditions.

As used herein, the term “plant” includes plant cells, plant protoplasts, plant cell tissue cultures from which watermelon plants can be regenerated, plant calli, plant clumps, and plant cells that are intact in plants or parts of plants, such as leaves, pollen, embryos, cotyledons, hypocotyl, roots, root tips, anthers, pistils, flowers, seeds, stems, stalks, fruits, and the like.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

### DEPOSIT INFORMATION

A deposit of at least 625 seeds of the watermelon variety ‘E26C.00181’ was made with the National Collection of Industrial, Food and Marine Bacteria Ltd. (NCIMB Ltd), Wellheads Place, Dyce, Aberdeen, AB21 7 GB, United Kingdom, and assigned NCIMB Number X1. The seeds deposited with the NCIMB on [DATE] were obtained from the seed of the variety maintained by Enza Zaden USA, Inc., 7 Harris Place, Salinas, California 93901, United States since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the watermelon variety ‘E26C.00181’ will be maintained in the NCIMB, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

1. A watermelon seed designated as ‘E26C.00181’, representative sample of seed having been deposited under NCIMB Accession Number X1.

2. A watermelon plant produced by growing the seed of claim 1.

3. A plant part from the plant of claim 2, wherein said part is a leaf, an ovule, a pollen grain, a fruit, a flower, a scion, a rootstock, a root, a root tip, a stem, a hypocotyl, a petiole, a cotyledon, a cutting, an anther, a pistil, a stalk, a meristem, a protoplast, or a cell.

4. The plant part of claim 3, wherein said part is a fruit.

5. A watermelon plant having all, or essentially all, of the physiological and morphological characteristics of the watermelon plant of claim 2.

6. A plant part from the plant of claim 5.

7. The plant part of claim 6, wherein said part is a leaf, an ovule, a pollen grain, a fruit, a flower, a scion, a rootstock, a root, a root tip, a stem, a hypocotyl, a petiole, a cotyledon, a cutting, an anther, a pistil, a stalk, a meristem, a protoplast, or a cell.

8. The plant part of claim 7, wherein said part is a fruit.

9. A pollen grain or an ovule of the plant of claim 2.

10. A protoplast produced from the plant of claim 2.

11. A tissue or cell culture produced from protoplasts or regenerable cells from the plant of claim 2.

12. The tissue or cell culture of claim 11, wherein said regenerable cells or protoplasts are produced from a plant part selected from the group consisting of root, root tip, meristematic cell, stem, hypocotyl, petiole, cotyledon, leaf, flower, anther, pollen, pistil, stalk, and fruit.

13. A watermelon plant regenerated from the tissue culture of claim 11, wherein the plant has all, or essentially all, of the morphological and physiological characteristics of a watermelon plant produced by growing watermelon seed designated as ‘E26C.00181’, representative sample of seed having been deposited under NCIMB Accession Number X1.

14. A method of producing seedless watermelon fruit, the method comprising:
(a) crossing the plant of claim 2 with a diploid watermelon plant;
(b) allowing seedless fruit to form; and
(c) harvesting the seedless fruit.

15. A method of vegetatively propagating a plant of watermelon variety ‘E26C.00181’, the method comprising the steps of:
(a) collecting tissue capable of being propagated from a plant of watermelon variety ‘E26C.00181’, representative seed of said watermelon variety ‘E26C.00181’ having been deposited under NCIMB Accession Number X1; and
(b) producing a rooted plant from said tissue.

16. A method of introducing a trait into watermelon variety ‘E26C.00181’, the method comprising: (a) utilizing as a recurrent parent the plant of claim 2 by crossing the recurrent parent plant with a donor plant that comprises the trait, thereby producing F1 progeny; (b) selecting an F1 progeny that comprises the trait; (c) backcrossing the selected F1 progeny with a plant of the same line used as the recurrent parent in step (a) to produce backcross progeny; (d) selecting a backcross progeny comprising the trait and otherwise comprising all or essentially all of the morphological and physiological characteristics of the recurrent parent plant; and (e) repeating steps (c) and (d) for at least one additional generation, thereby producing further backcross progeny.

17. The method of claim 16, wherein the at least one additional generation comprises 3-10 generations.

18. A watermelon plant produced by the method of claim 16, wherein the plant comprises the trait and otherwise comprises all or essentially all of the morphological and physiological characteristics of watermelon variety ‘E26C.00181’, or a seed thereof.

19. A method of producing a watermelon plant comprising an added trait, the method comprising introducing a transgene conferring the trait into the plant of claim 2.

20. A watermelon plant produced by the method of claim 19, wherein the plant comprises the trait and otherwise comprises all or essentially all of the morphological and physiological characteristics of watermelon variety ‘E26C.00181’, or a seed thereof.

21. A watermelon plant having all or essentially all of the morphological and physiological characteristics of watermelon variety ‘E26C.00181’, representative sample of seed having been deposited under NCIMB Accession Number X1, and further comprising a transgene.
